(19) United States
(12) Patent Application Publication
(10) Pub. No.: US 2021/0059523 A1
(43) Pub. Date: Mar. 4, 2021

(54) DETACHABLE MULTI-FUNCTIONAL OPHTHALMIC TESTER

(71) Applicant: Crystalvue Medical Corporation, Taoyuan City (TW)

(72) Inventors: Wen-Wei HUANG, Taoyuan City (TW); Kuan-Heng WU, Taoyuan City (TW); Chao-Ching LIN, Taoyuan City (TW)

(21) Appl. No.: 17/002,668

(22) Filed: Aug. 25, 2020

Related U.S. Application Data
(60) Provisional application No. 62/892,302, filed on Aug. 27, 2019.

(51) Int. Cl. A61B 3/12; A61B 3/10; A61B 3/00

(52) U.S. Cl. CPC A61B 3/12; A61B 3/0083; A61B 3/0075; A61B 3/102

(57) ABSTRACT

A detachable multi-functional ophthalmic tester is disclosed. The detachable multi-functional ophthalmic tester includes a movable platform and a measuring module. The movable platform has a first connecting portion and it can move along at least one direction. The measuring module is coupled to the first connecting portion. The measuring module is used to perform measurement on the eye to be tested. The movable platform and the measuring module are both detachable. The movable platform can move along X-axis direction, Y-axis direction and Z-axis direction. The first connecting portion of the movable platform can connect with different measuring modules to provide different functions. The measuring module can connect with different movable platforms.

### CROSS REFERENCE TO RELATED APPLICATIONS

This Application is a non-provisional application claiming priority to U.S. Provisional Application 62/892,302 filed on Aug. 27, 2019, which is incorporated by reference in its entirety.

### BACKGROUND OF THE INVENTION

### Field of the Invention

The invention relates to ophthalmic test; in particular, to a detachable multi-functional ophthalmic tester.

### Description of the Prior Art

Please refer to FIG. 1A, since the volume of the conventional fixed ophthalmic tester 1 is usually quite large, if the conventional fixed ophthalmic tester 1 needs to be used in different places, the user P needs to accommodate the conventional fixed ophthalmic tester 1 in a large box BX having the size several times of the normal suitcase and needs to use the cart to transport it, which is quite inconvenient and laborious for the user.

Please refer to FIG. 1B, the conventional fixed ophthalmic tester 1 usually needs to be fixedly set on a desktop for use. When the user P uses the conventional fixed ophthalmic tester 1, he/she needs to align his/her eyes with the height of the camera of the conventional fixed ophthalmic tester 1 as much as possible to facilitate the ophthalmic test. However, since each user's height and eye position are not the same, the above-mentioned alignment procedure may not be easily completed, which often causes considerable inconvenience and trouble to the user.

In addition, the conventional fixed ophthalmic tester 1 usually only provides a single ophthalmic testing function. If the user wants to user multiple ophthalmic testing functions, multiple fixed ophthalmic testers with different ophthalmic testing functions should be set up respectively. It will greatly increase the cost, and it will take up a lot of space.

From the above, it can be seen that the conventional fixed ophthalmic tester 1 still has many problems in practical application, which need to be overcome urgently.

### SUMMARY OF THE INVENTION

Therefore, the invention provides a detachable multi-functional ophthalmic tester to solve the above-mentioned problems of the prior arts.

A preferred embodiment of the invention is a detachable multi-functional ophthalmic tester. In this embodiment, the detachable multi-functional ophthalmic tester includes a movable platform and a measuring module. The movable platform has a first connecting portion and it can move along at least one direction. The measuring module is coupled to the first connecting portion. The measuring module is used to perform measurement on the eye to be tested. The movable platform and the measuring module are both detachable.

In an embodiment, the detachable multi-functional ophthalmic tester also includes a chin holding module, which is coupled to a second connecting portion of the movable platform to hold a chin of a user.

In an embodiment, the chin holding module is detachable.

In an embodiment, the detachable multi-functional ophthalmic tester also includes a forehead holding module, which is coupled to a third connecting portion of the movable platform to hold a forehead of a user.

In an embodiment, the forehead holding module is detachable.

In an embodiment, in a measurement mode, the forehead holding module is erected on the movable platform to correspond to the forehead of the user.

In an embodiment, in a storage mode, the forehead holding module is lying flat on the movable platform to save space and facilitate storage.

In an embodiment, the measuring module is a fundus camera including a camera for performing fundus imaging on the eye to be tested.

In an embodiment, the measuring module is a tonometer or an optical coherence tomography (OCT) scanner.

In an embodiment, the movable platform moves along directions of X-axis, Y-axis and Z-axis.

In an embodiment, the operation of the detachable multi-function ophthalmic tester is controlled by a remote signal from a mobile communication device.

In an embodiment, the detachable multi-functional ophthalmic tester is placed on a liftable platform, and the liftable platform raises or lowers its height to make the measuring module to be aligned with the eye to be tested.

In an embodiment, the measuring module is placed on a liftable platform, and the liftable platform raises or lowers its height to make the measuring module to be aligned with the eye to be tested.

In an embodiment, the first connecting portion of the movable platform is coupled to different measuring modules to provide different measuring functions.

In an embodiment, the measuring module is coupled to different movable platforms.

Compared to the prior art, the detachable multi-functional ophthalmic tester of the invention not only can be stored in a general suitcase for transportation, but also can adjust the height and position of the camera according to the height and eye position of each user, and select different measuring modules to provide different measuring functions. For the users, the convenience of transportation and operation can be greatly increased, and the cost and space of installing multiple ophthalmic testers can also be saved.

The advantage and spirit of the invention may be understood by the following detailed descriptions together with the appended drawings.

### BRIEF DESCRIPTION OF THE APPENDED DRAWINGS

FIG. 1A is a schematic diagram showing that the user P transports the conventional fixed ophthalmic tester 1 accommodated in the large-volume box BX through the cart.

FIG. 1B is a schematic diagram showing that the conventional fixed ophthalmic tester 1 is fixedly arranged on the desktop.

FIG. 2 is a schematic diagram of the detachable multi-functional ophthalmic tester 2 in a preferred embodiment of the invention.

FIG. 2A to FIG. 2C are schematic diagrams showing the measuring module 20, the movable platform 22, the forehead holding module 26 and the chin holding module 24 in the detachable multi-functional ophthalmic tester 2 respectively.

FIG. 3A is a schematic diagram showing that the measuring module 20 of the detachable multi-functional ophthalmic tester 2 can be aligned with the eye to be tested by adjusting the height of the liftable platform 30.

FIG. 3B is a schematic diagram showing that the measuring module 20 can be aligned with the eye to be tested by adjusting the height of the liftable platform 30 and 32.

FIG. 4 is a schematic diagram showing that the operation of the detachable multi-functional ophthalmic tester 2 controlled by the remote signal RM sent by the mobile communication device MP.

FIG. 5 is a schematic diagram showing the forehead holding module 26 lying flat on the movable platform 22 in the storage mode to save space and facilitate storage.

FIG. 6A is a schematic diagram showing that the measuring module 20, the movable platform 22 and the forehead holding module 26 of the detachable multi-functional ophthalmic tester 2 is accommodated in the ordinary suitcase SC.

FIG. 6B is a schematic diagram showing that the user P can easily pull the suitcase SC to transport the detachable multi-functional ophthalmic tester 2.

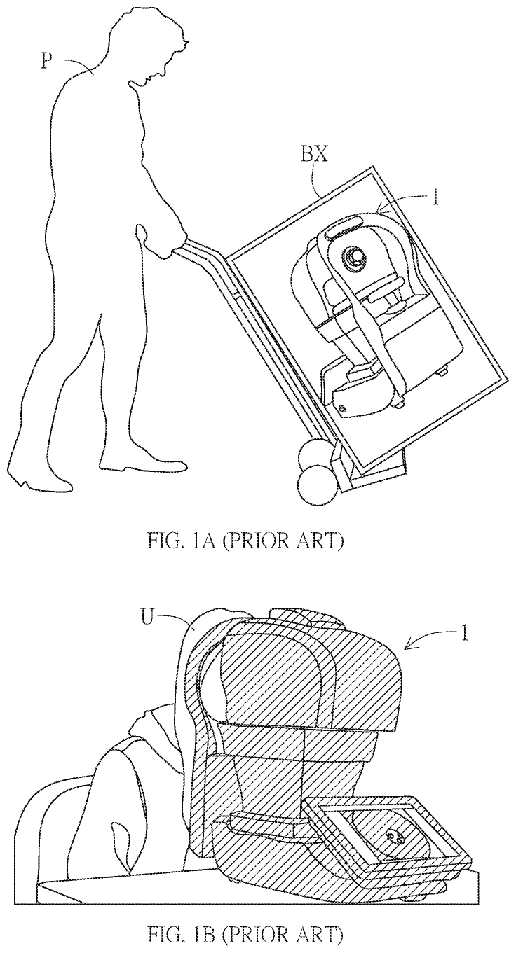

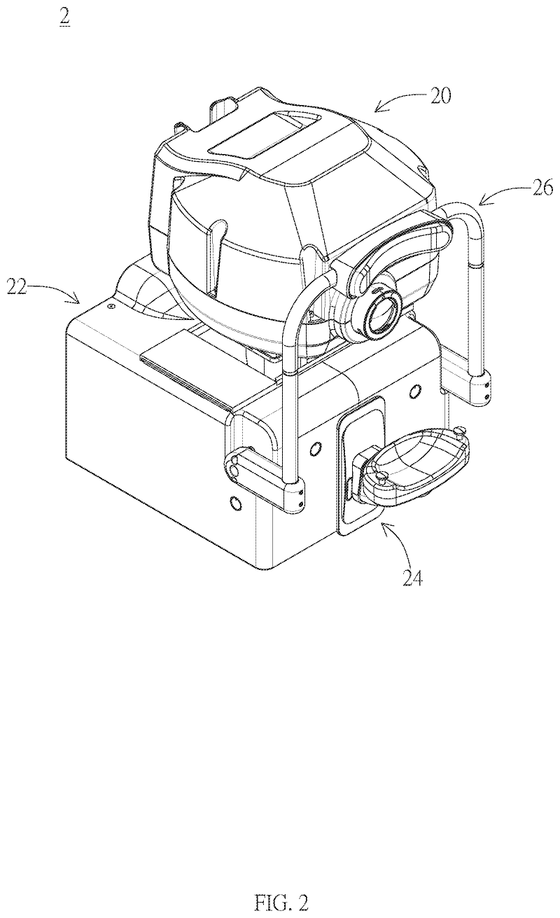

### DETAILED DESCRIPTION OF THE INVENTION

Exemplary embodiments of the invention are referenced in detail now, and examples of the exemplary embodiments are illustrated in the drawings. Further, the same or similar reference numerals of the components/components in the drawings and the detailed description of the invention are used on behalf of the same or similar parts.

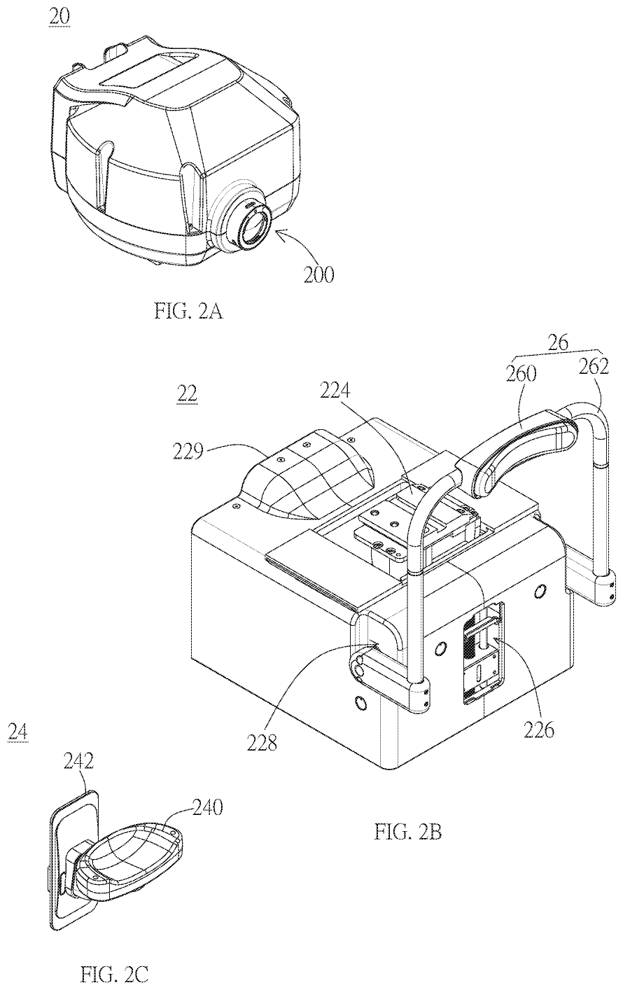

According to a specific embodiment of the invention, a detachable multi-functional ophthalmic tester. Please refer to FIG. 2. FIG. 2 is a schematic diagram of the detachable multi-functional ophthalmic tester 2 in this embodiment.

As shown in FIG. 2, the detachable multi-functional ophthalmic tester 2 can include a measuring module 20, a movable platform 22, a chin holding module 24 and a forehead holding module 26. It should be noted that the measuring module 20, the movable platform 22, the chin holding module 24 and the forehead holding module 26 in the detachable multi-functional ophthalmic tester 2 are all detachable, and the measuring module 20, the chin holding module 24 and the forehead holding module 26 can be coupled to the movable platform 22 respectively, but not limited to this.

In practical applications, the movable platform 22 can be coupled to different measuring modules according to practical needs, such as: fundus camera, tonometer, or optical coherence tomography (OCT) scanner, etc., to provide different measuring functions for the eye to be tested. In contrast, the measuring module 20 can also be coupled to different movable platforms according to practical needs, so as to be provided to different locations or different users.

Next, please refer to FIG. 2A to FIG. 2C respectively. FIG. 2A to FIG. 2C are schematic diagrams showing the measuring module 20, the movable platform 22, the forehead holding module 26 and the chin holding module 24 in the detachable multi-functional ophthalmic tester 2 respectively.

The movable platform 22 includes a first connecting portion 224, a second connecting portion 226 and a third connecting portion 228 for connecting the measuring module 20, the chin holding module 24 and the forehead holding module 26 respectively, but not limited to this. In fact, there is no specific limitations to the way that the first connecting portion 224, the second connecting portion 226 and the third connecting portion 228 of the movable platform 22 are coupled to the measuring module 20, the chin holding module 24 and the forehead holding module 26 respectively.

For example, the first connecting portion 224 of the movable platform 22 and the measuring module 20 can be coupled by snapping; the second connecting portion 226 of the movable platform 22 and the chin holding module 24 can be coupled by locking; the third connecting portion 228 of the movable platform 22 and the forehead holding module 26 can be coupled by locking, but not limited to this.

The movable platform 22 can also be referred to as a tracking module, which can move in at least one direction, for example, along the X-axis direction, the Y-axis direction and/or the Z-axis direction, but not limited to this. In other words, the movable platform 22 can be moved in one, two or three dimensions according to actual needs. In addition, the movable platform 22 can also be provided with a buffer 229. In the storage mode, the forehead holding module 26 lies flat on the buffer portion 229 of the movable platform 22, but it is not limited to this.

In practical applications, the measuring module 20 can be a fundus camera, a tonometer or an optical coherence tomography (OCT) scanner, but not limited to this. For example, if the measuring module 20 is a fundus camera, then the measuring module 20 can include a camera 200 for performing fundus imaging on the eye to be tested.

The chin holding module 24 includes a chin holder 240 and a fourth connecting portion 242. The chin holder 240 is disposed on the fourth connecting portion 242. The fourth connecting portion 242 can be coupled to the second connecting portion 226. When the movable platform 22 moves, the second connecting portion 226 of the movable platform 22 brings the fourth connecting portion 242 of the chin holding module 24 to move, so that the position of the chin holder 240 disposed on the fourth connecting portion 242 can correspond to the user's chin and hold it.

The forehead holding module 26 includes a forehead holder 260 and a fifth connecting portion 262. The forehead holder 260 is disposed on the fifth connecting portion 262. The fifth connecting portion 262 can be coupled to the third connecting portion 228. When the movable platform 22 moves, the third connecting portion 228 of the movable platform 22 brings the fifth connecting portion 262 of the forehead holding module 26 to move, so that the forehead holder 260 disposed on the fifth connecting portion 262 can correspond to the user's forehead and hold it.

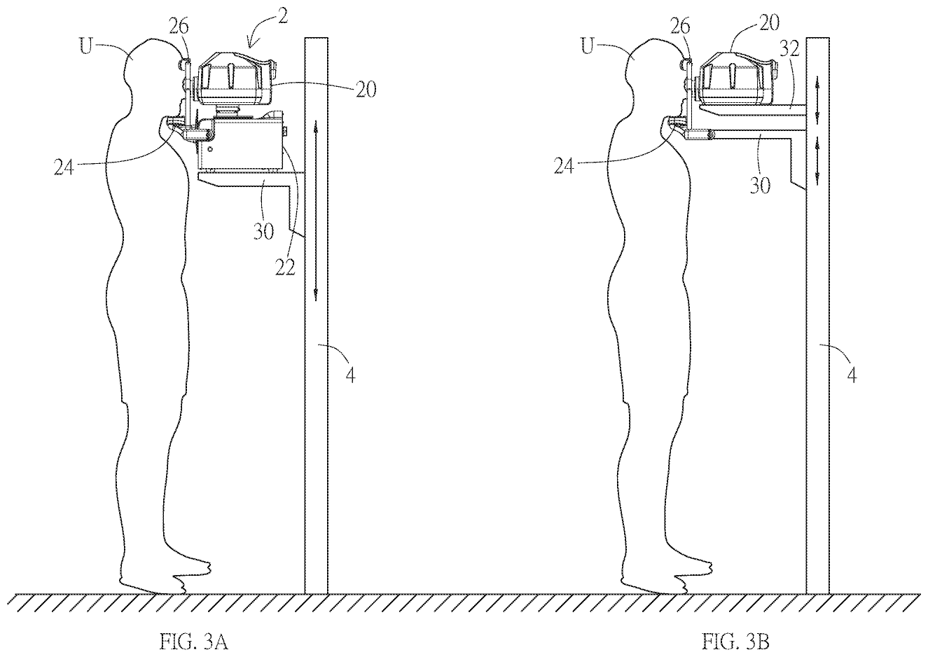

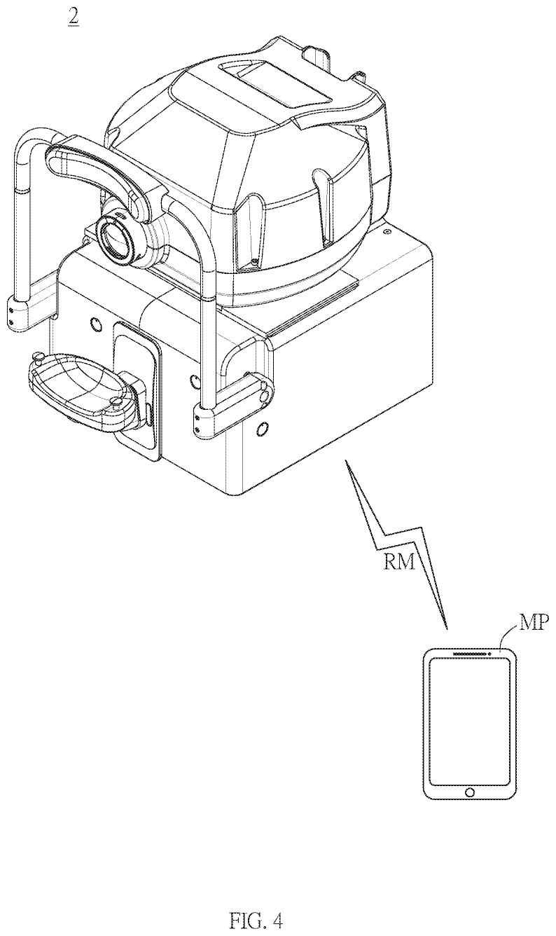

Please refer to FIG. 3A. In an embodiment, the detachable multi-functional ophthalmic tester 2 can be placed on the liftable platform 30. The liftable platform 30 can be set on the wall 4 and can move up and down along the wall 4. Therefore, the operator can adjust the height of the liftable platform 30, so that the measuring module 20 of the detachable multi-functional ophthalmic tester 2 placed on the liftable platform 30 can be aligned with the eye to be tested of the user U, and the chin holding module 24 and the forehead holding module 26 hold the chin and forehead of the user U respectively to facilitate the measurement by the measuring module 20.

Please refer to FIG. 3B. In another embodiment, the measuring module 20 can be placed on the liftable platform 32, and the chin holding module 24 and the forehead holding module 26 can be placed on the liftable platform 30. The liftable platforms 30 and 32 can be arranged on the wall 4 and can move up and down along the wall 4. Therefore, the operator can adjust the height of the liftable platforms 30 and 32, so that the measuring module 20 placed on the liftable platform 32 can be aligned with the eye to be tested of the user U, and the chin holding module 24 and the forehead holding module 26 placed on the liftable platform 30 hold the chin and forehead of the user U respectively to facilitate the measurement by the measuring module 20.

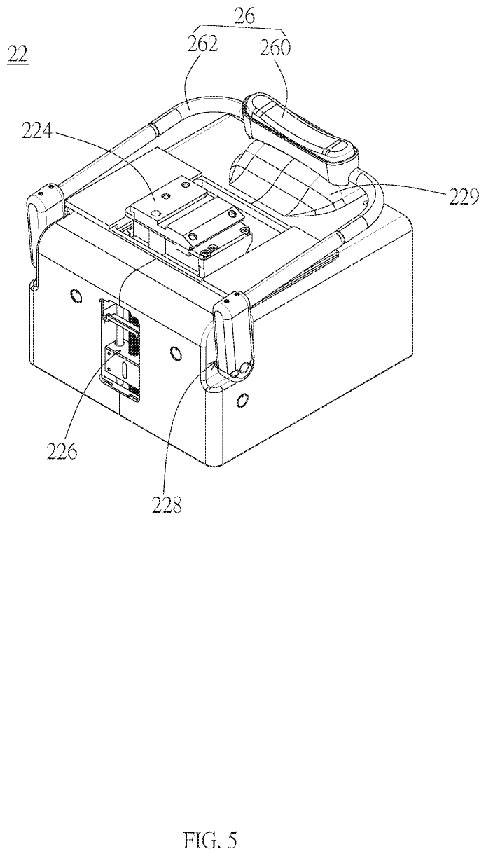

Please refer to FIG. 4, in another embodiment, the user can send a remote signal RM to the detachable multi-functional ophthalmic tester 2 by operating the mobile communication device MP (such as a smart phone), thereby controlling the operation of the detachable multi-functional ophthalmic tester 2, but not limited to this.

In practical applications, the forehead holding module 26 is erected on the movable platform 22 in the measuring mode to hold the forehead of the user U, as shown in FIG. 3A. In addition, the forehead holding module 26 lies flat on the buffer portion 229 of the movable platform 22 in the storage mode to save space and facilitate storage, as shown in FIG. 5.

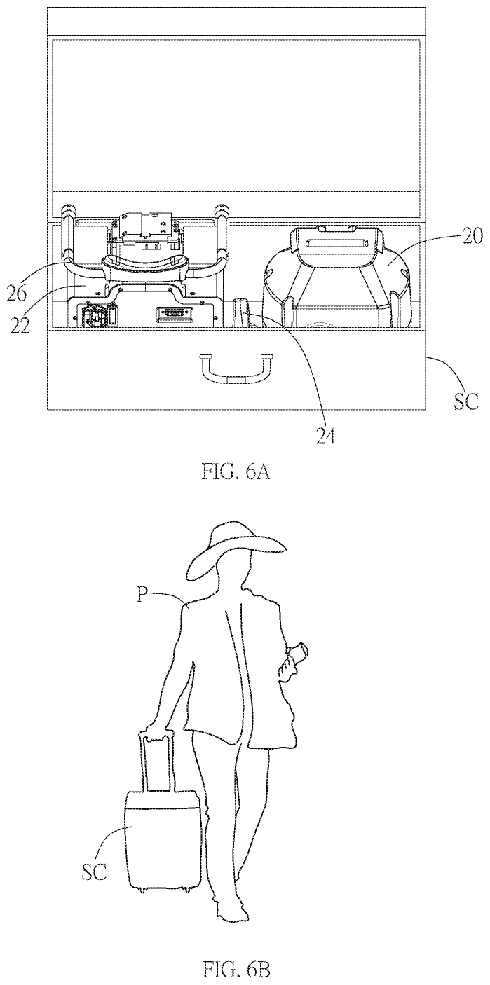

Please refer to FIG. 6A and FIG. 6B. After the measuring module 20, the movable platform 22, the chin holding module 24, and the forehead holding module 26 in the detachable multi-functional ophthalmic tester 2 of the invention are removed, it can be accommodated together in a suitcase SC of a general size (for example, 20 inches). Therefore, the user P can easily pull the suitcase SC to bring the measuring module 20, the movable platform 22, the chin holding module 24 and the forehead holding module 26 to other locations, and then the user P can assemble them into the detachable multi-functional ophthalmic tester 2 for testing, which is quite convenient and labor-saving.

Compared to the prior art, the detachable multi-functional ophthalmic tester of the invention not only can be stored in a general suitcase for transportation, but also can adjust the height and position of the camera according to the height and eye position of each user, and select different measuring modules to provide different measuring functions. For the users, the convenience of transportation and operation can be greatly increased, and the cost and space of installing multiple ophthalmic testers can also be saved.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A detachable multi-functional ophthalmic tester, comprising:
a movable platform having a first connecting portion and capable of moving along at least one direction; and
a measuring module, coupled to the first connecting portion, performing measurement on the eye to be tested;
wherein the movable platform and the measuring module are both detachable.

2. The detachable multi-functional ophthalmic tester of claim 1, further comprising:
a chin holding module, coupled to a second connecting portion of the movable platform to hold a chin of a user.

3. The detachable multi-functional ophthalmic tester of claim 2, wherein the chin holding module is detachable.

4. The detachable multi-functional ophthalmic tester of claim 1, further comprising:
a forehead holding module, coupled to a third connecting portion of the movable platform to support a forehead of a user.

5. The detachable multi-functional ophthalmic tester of claim 4, wherein the forehead holding module is detachable.

6. The detachable multi-functional ophthalmic tester of claim 4, wherein the forehead holding module is erected on the movable platform to correspond to the forehead of the user.

7. The detachable multi-functional ophthalmic tester of claim 4, wherein in a storage mode, the forehead holding module is lying flat on the movable platform to save space and facilitate storage.

8. The detachable multi-functional ophthalmic tester of claim 1, wherein the measuring module is a fundus camera comprising a camera for performing fundus imaging on the eye to be tested.

9. The detachable multi-functional ophthalmic tester of claim 1, wherein the measuring module is a tonometer or an optical coherence tomography scanner.

10. The detachable multi-functional ophthalmic tester of claim 1, wherein the movable platform moves along directions of X-axis, Y-axis and Z-axis.

11. The detachable multi-functional ophthalmic tester of claim 1, wherein the operation of the detachable multi-function ophthalmic tester is controlled by a remote signal from a mobile communication device.

12. The detachable multi-functional ophthalmic tester of claim 1, wherein the detachable multi-functional ophthalmic tester is placed on a liftable platform, and the liftable platform raises or lowers its height to make the measuring module to be aligned with the eye to be tested.

13. The detachable multi-functional ophthalmic tester of claim 1, wherein the measuring module is placed on a liftable platform, and the liftable platform raises or lowers its height to make the measuring module to be aligned with the eye to be tested.

14. The detachable multi-functional ophthalmic tester of claim 1, wherein the first connecting portion of the movable platform is coupled to different measuring modules to provide different measuring functions.

15. The detachable multi-functional ophthalmic tester of claim 1, wherein the measuring module is coupled to different movable platforms.
